(19) United States
(12) Patent Application Publication
(10) Pub. No.: US 2019/0328251 A1
(43) Pub. Date: Oct. 31, 2019

(54) ARRHYTHMIA DETECTION METHOD, ARRHYTHMIA DETECTION DEVICE AND ARRHYTHMIA DETECTION SYSTEM

(71) Applicant: BOE TECHNOLOGY GROUP CO., LTD., Beijing (CN)

(72) Inventor: Hailan Jin, Beijing (CN)

(21) Appl. No.: 16/364,040

(22) Filed: Mar. 25, 2019

(30) Foreign Application Priority Data
Apr. 27, 2018 (CN) 201810395606.8

(51) Int. Cl. A61B 5/046; A61B 5/0464; A61B 5/00

(52) U.S. Cl. CPC A61B 5/046; A61B 5/0456; A61B 5/7264; A61B 5/0464

(57) ABSTRACT

An arrhythmia detection method, an arrhythmia detection device and an arrhythmia detection system are provided. The arrhythmia detection device comprises a processor to: equally divide an electrocardiogram signal through each time interval to acquire respective sequences of electrocardiogram signal segments; determine an arrhythmia probability for each electrocardiogram signal segment in each of the sequences of electrocardiogram signal segments; and for each two adjacent time intervals, determine an arrhythmia detection result for each electrocardiogram signal segment in the sequence of electrocardiogram signal segments corresponding to a shorter one of the time intervals in accordance with the arrhythmia probability for each electrocardiogram signal segment in the sequence of electrocardiogram signal segments corresponding to the shorter one of the time intervals, and the arrhythmia probability or an arrhythmia detection result for each electrocardiogram signal segment in the sequence of electrocardiogram signal segments corresponding to a longer one of the time intervals.

### CROSS-REFERENCE TO RELATED APPLICATION

The present application claims a priority of the Chinese patent application No. 201810395606.8 filed on Apr. 27, 2018, which is incorporated herein by reference in its entirety.

### TECHNICAL FIELD

The present disclosure relates to the field of medical equipment, in particular to a physiological signal rhythm detection method and a physiological signal rhythm detection system, more particularly to an arrhythmia detection method, an arrhythmia detection device, and an arrhythmia detection system.

### BACKGROUND

As a periodical physiological electric signal, an electrocardiogram signal may be adopted to reflect a heart function. Electrocardiogram monitoring is an important means for evaluating a health condition of a cardiovascular system. With the development of technology, it is possible to perform long-term electrocardiogram monitoring. Due to the aging of the population, a prevalence rate of disease of the cardiovascular system increases, and for each individual, the monitoring and analysis of the electrocardiogram signal plays a very important role in the diagnosis and prevention of disease of the cardiovascular system.

A periodical waveform feature, especially a rhythm feature, of the electrocardiogram signal may be adopted to reflect various structural and functional disorders of the heart. Usually, arrhythmias may be classified in accordance with body parts and levels of risk. Based on the body parts, the arrhythmias may include sinus arrhythmia, junctional arrhythmia (occurring at an atrioventricular junction), atrial arrhythmia, ventricular arrhythmia, and so on. Based on the levels of risk, the arrhythmias may include arrhythmia which is not seriously harmful to health and for which no medical treatment is required (e.g., a pure and slight sinus arrhythmia), and arrhythmia which is harmful to health and for which medical treatment is required (including fatal arrhythmia, e.g., auricular fibrillation, tachycardia-type auricular flutter, ventricular tachycardia, ventricular fibrillation, ventricular flutter, and three-degree atrioventricular block).

Auricular fibrillation (AF) is the most common perpetual arrhythmia. The incidence of AF for each individual increases gradually with age, and for people over the age of 75, the incidence of AF may be up to 10%. During AF, an auricular activation frequency is 300 to 600 times per minute, a heartbeat frequency is usually too fast and irregular, and sometimes it may be 100 to 160 times per minute. This heartbeat frequency is much faster than a heartbeat frequency of a healthy individual, and seriously disordered, so an atrium may lose its effective contraction function. Although less common than AF, auricular flutter and the ventricular fibrillation are also highly risky. Especially, ventricular fibrillation is extremely fatal.

### SUMMARY

In one aspect, the present disclosure provides in some embodiments an arrhythmia detection method, including: acquiring an electrocardiogram signal; setting a group of time intervals with gradually increasing lengths, wherein for each two adjacent time intervals in the group of time intervals, a length of a latter one time interval is an integral multiple of a length of a former one time interval; equally dividing the acquired electrocardiogram signal through each of the time intervals to acquire respective sequences of electrocardiogram signal segments, wherein each of the time intervals in the group of time intervals corresponds to a respective one sequence of electrocardiogram signal segments; determining an arrhythmia probability for each electrocardiogram signal segment in each of the sequences of electrocardiogram signal segments; and for respective two sequences of electrocardiogram signal segments corresponding to each two adjacent time intervals, determining an arrhythmia detection result for each electrocardiogram signal segment in the sequence of electrocardiogram signal segments corresponding to a shorter one of the time intervals in accordance with the arrhythmia probability for the electrocardiogram signal segment in the sequence of electrocardiogram signal segments corresponding to the shorter one of the time intervals, and the arrhythmia probability or an arrhythmia detection result for a respective electrocardiogram signal segment in the sequence of electrocardiogram signal segments corresponding to a longer one of the time intervals, wherein the respective electrocardiogram signal segment in the sequence of electrocardiogram signal segments corresponding to a longer one of the time intervals comprises the electrocardiogram signal segment in the sequence of electrocardiogram signal segments corresponding to the shorter one of the time intervals.

In some embodiments of the present disclosure, for each two adjacent time intervals, a start position of each electrocardiogram signal segment in the sequence of electrocardiogram signal segments corresponding to the longer one of the time intervals is aligned with a start position of a respective one electrocardiogram signal segment in the sequence of electrocardiogram signal segments corresponding to the shorter one of the time intervals.

In some embodiments of the present disclosure, the arrhythmia detection result includes a label indicating an arrhythmia condition.

In some embodiments of the present disclosure, for respective two sequences of electrocardiogram signal segments corresponding to each two adjacent time intervals, the determining the arrhythmia detection result for each electrocardiogram signal segment in the sequence of electrocardiogram signal segments corresponding to the shorter one of the time intervals comprises: when the longer one of the time intervals is a maximum time interval in the groups of time intervals, determining the arrhythmia detection result for each electrocardiogram signal segment in the sequence of electrocardiogram signal segments corresponding to the shorter one of the time intervals in accordance with the arrhythmia probability for the electrocardiogram signal segment in the sequence of electrocardiogram signal segments corresponding to the shorter one of the time intervals, and the arrhythmia probability for the respective electrocardiogram signal segment in the sequence of electrocardiogram signal segments corresponding to the longer one of the time intervals, wherein the respective electrocardiogram signal segment in the sequence of electrocardiogram signal segments corresponding to the longer one of the time intervals comprises the electrocardiogram signal segment in the sequence of electrocardiogram signal segments corresponding to the shorter one of the time intervals, and when the longer one of the time intervals is not the maximum time interval in the groups of time intervals, determining the arrhythmia detection result for each electrocardiogram signal segment in the sequence of electrocardiogram signal segments corresponding to the shorter one of the time intervals in accordance with the arrhythmia probability for the electrocardiogram signal segment in the sequence of electrocardiogram signal segments corresponding to the shorter one of the time intervals, and the arrhythmia detection result for the respective electrocardiogram signal segment in the sequence of electrocardiogram signal segments corresponding to the longer one of the time intervals, wherein the respective electrocardiogram signal segment in the sequence of electrocardiogram signal segments corresponding to the longer one of the time intervals comprises the electrocardiogram signal segment in the sequence of electrocardiogram signal segments corresponding to the shorter one of the time intervals.

In some embodiments of the present disclosure, the electrocardiogram signal segments in each sequence of electrocardiogram signal segments are consecutive.

In another aspect, the present disclosure provides in some embodiments an arrhythmia detection device, including a memory, a processor, and a computer-executable instruction stored in the memory and executed by the processor. The processor is configured to execute the computer-executable instruction to: set a group of time intervals with gradually increasing lengths, wherein for each two adjacent time intervals in the group of time intervals, a length of a latter one time interval is an integral multiple of a length of a former one time interval; equally divide an electrocardiogram signal through each of the time intervals to acquire respective sequences of electrocardiogram signal segments, wherein each of the time intervals in the group of time intervals corresponds to a respective one sequence of electrocardiogram signal segments; determine an arrhythmia probability for each electrocardiogram signal segment in each of the sequences of electrocardiogram signal segments; and for respective two sequences of electrocardiogram signal segments corresponding to each two adjacent time intervals, determine an arrhythmia detection result for each electrocardiogram signal segment in the sequence of electrocardiogram signal segments corresponding to a shorter one of the time intervals in accordance with the arrhythmia probability for each electrocardiogram signal segment in the sequence of electrocardiogram signal segments corresponding to the shorter one of the time intervals, and the arrhythmia probability or an arrhythmia detection result for each respective electrocardiogram signal segment in the sequence of electrocardiogram signal segments corresponding to a longer one of the time intervals, wherein each respective electrocardiogram signal segment in the sequence of electrocardiogram signal segments corresponding to a longer one of the time intervals comprises the electrocardiogram signal segment in the sequence of electrocardiogram signal segments corresponding to the shorter one of the time intervals.

In some embodiments of the present disclosure, for each two adjacent time intervals, a start position of each electrocardiogram signal segment in the sequence of electrocardiogram signal segments corresponding to the longer one of the time intervals is aligned with a start position of a respective one electrocardiogram signal segment in the sequence of electrocardiogram signal segments corresponding to the shorter one of the time intervals.

In some embodiments of the present disclosure, the arrhythmia detection result includes a label indicating an arrhythmia condition.

In some embodiments of the present disclosure, the processor is further configured to execute the computer-executable instructions to, for respective two sequences of electrocardiogram signal segments corresponding to each two adjacent time intervals, when the longer one of the time intervals is a maximum time interval in the groups of time intervals, determine the arrhythmia detection result for each electrocardiogram signal segment in the sequence of electrocardiogram signal segments corresponding to the shorter one of the time intervals in accordance with the arrhythmia probability for the electrocardiogram signal segment in the sequence of electrocardiogram signal segments corresponding to the shorter one of the time intervals, and the arrhythmia probability for the respective electrocardiogram signal segment in the sequence of electrocardiogram signal segments corresponding to the longer one of the time intervals, wherein the respective electrocardiogram signal segment in the sequence of electrocardiogram signal segments corresponding to the longer one of the time intervals comprises the electrocardiogram signal segment in the sequence of electrocardiogram signal segments corresponding to the shorter one of the time intervals, and when the longer one of the time intervals is not the maximum time interval in the groups of time intervals, determine the arrhythmia detection result for each electrocardiogram signal segment in the sequence of electrocardiogram signal segments corresponding to the shorter one of the time intervals in accordance with the arrhythmia probability for the electrocardiogram signal segment in the sequence of electrocardiogram signal segments corresponding to the shorter one of the time intervals, and the arrhythmia detection result for the respective electrocardiogram signal segment in the sequence of electrocardiogram signal segments corresponding to the longer one of the time intervals, wherein the respective electrocardiogram signal segment in the sequence of electrocardiogram signal segments corresponding to the longer one of the time intervals comprises the electrocardiogram signal segment in the sequence of electrocardiogram signal segments corresponding to the shorter one of the time intervals.

In some embodiments of the present disclosure, the electrocardiogram signal segments in each sequence of electrocardiogram signal segments are consecutive.

In some embodiments of the present disclosure, the electrocardiogram signal segments in at least one of the sequences of electrocardiogram signal segments overlap each other.

In some embodiments of the present disclosure, the processor is further configured to execute the computer-executable instructions to, for each electrocardiogram signal segment in the sequence of electrocardiogram signal segments corresponding to the longer one of the time intervals in the respective two sequences of electrocardiogram signal segments corresponding to each two adjacent time intervals, when the arrhythmia probability for the electrocardiogram signal segment is greater than or equal to a threshold, or when the arrhythmia detection result for the electrocardiogram signal segment is positive, and the arrhythmia probability for at least one of the electrocardiogram signal segments in the sequence of electrocardiogram signal segments corresponding to the shorter one of the time intervals comprised in the electrocardiogram signal segment in the sequence of electrocardiogram signal segments corresponding to the longer one of the time intervals is greater than the threshold, determine the arrhythmia detection result for each electrocardiogram signal segment in the sequence of electrocardiogram signal segments corresponding to the shorter one of the time intervals comprised in the electrocardiogram signal segment in the sequence of electrocardiogram signal segments corresponding to the longer one of the time intervals to be positive.

In some embodiments of the present disclosure, the processor is further configured to execute the computer-executable instructions to, for each electrocardiogram signal segment in the sequence of electrocardiogram signal segments corresponding to the longer one of the time intervals in the respective two sequences of electrocardiogram signal segments corresponding to each two adjacent time intervals, when the arrhythmia probability for the electrocardiogram signal segment is greater than or equal to a threshold, or when the arrhythmia detection result for the electrocardiogram signal segment is positive, and the arrhythmia probability for each electrocardiogram signal segment in the sequence of electrocardiogram signal segments corresponding to the shorter one of the time intervals comprised in the electrocardiogram signal segment in the sequence of electrocardiogram signal segments corresponding to the longer one of the time intervals is smaller than the threshold, determine the arrhythmia detection result for at least one electrocardiogram signal segment, but not all the electrocardiogram signal segments, in the sequence of electrocardiogram signal segments corresponding to the shorter one of the time intervals comprised in the electrocardiogram signal segment in the sequence of electrocardiogram signal segments corresponding to the longer one of the time intervals to be positive.

In some embodiments of the present disclosure, the processor is further configured to execute the computer-executable instructions to, for each electrocardiogram signal segment in the sequence of electrocardiogram signal segments corresponding to the longer one of the time intervals in the respective two sequences of electrocardiogram signal segments corresponding to each two adjacent time intervals, when the arrhythmia probability for the electrocardiogram signal segment is smaller than a threshold, or when the arrhythmia detection result for the electrocardiogram signal segment is negative, and the arrhythmia probability for at least one electrocardiogram signal segment in the sequence of electrocardiogram signal segments corresponding to the shorter one of the time intervals comprised in the electrocardiogram signal segment in the sequence of electrocardiogram signal segments corresponding to the longer one of the time intervals is smaller than the threshold, determine the arrhythmia detection result for each electrocardiogram signal segment in the sequence of electrocardiogram signal segments corresponding to the shorter one of the time intervals comprised in the electrocardiogram signal segment in the sequence of electrocardiogram signal segments corresponding to the longer one of the time intervals to be negative.

In some embodiments of the present disclosure, the processor is further configured to execute the computer-executable instructions to, for each electrocardiogram signal segment in the sequence of electrocardiogram signal segments corresponding to the longer one of the time intervals in the respective two sequences of electrocardiogram signal segments corresponding to each two adjacent time intervals, when the arrhythmia probability for the electrocardiogram signal segment is smaller than a threshold, or when the arrhythmia detection result for the electrocardiogram signal segment is negative, and the arrhythmia probability for each electrocardiogram signal segment in the sequence of electrocardiogram signal segments corresponding to the shorter one of the time intervals comprised in the electrocardiogram signal segment in the sequence of electrocardiogram signal segments corresponding to the longer one of the time intervals is greater than or equal to the threshold, determine the arrhythmia detection result for at least one electrocardiogram signal segment, but not all the electrocardiogram signal segments, in the sequence of electrocardiogram signal segments corresponding to the shorter one of the time intervals comprised in the electrocardiogram signal segment in the sequence of electrocardiogram signal segments corresponding to the longer one of the time intervals to be negative.

In some embodiments of the present disclosure, the processor is further configured to execute the computer-executable instructions to, for each electrocardiogram signal segment in the sequence of electrocardiogram signal segments corresponding to the shorter one of the time intervals in the respective two sequences of electrocardiogram signal segments corresponding to each two adjacent time intervals, when each of the arrhythmia probabilities for at least two electrocardiogram signal segments in the sequence of electrocardiogram signal segments corresponding to the longer one of the time intervals comprising the electrocardiogram signal segment in the sequence of electrocardiogram signal segments corresponding to the shorter one of the time intervals is greater than or equal to a threshold, or when the arrhythmia detection result for the electrocardiogram signal segment in the sequence of electrocardiogram signal segments corresponding to the shorter one of the time intervals is positive, determine the arrhythmia detection result for the electrocardiogram signal segment in the sequence of electrocardiogram signal segments corresponding to the shorter one of the time intervals to be positive.

In some embodiments of the present disclosure, wherein the processor is further configured to execute the computer-executable instructions to, for each electrocardiogram signal segment in the sequence of electrocardiogram signal segments corresponding to the shorter one of the time intervals in the respective two sequences of electrocardiogram signal segments corresponding to each two adjacent time intervals, when each of the arrhythmia probabilities for at least two electrocardiogram signal segments in the sequence of electrocardiogram signal segments corresponding to the longer one of the time intervals comprising the electrocardiogram signal segment in the sequence of electrocardiogram signal segments corresponding to the shorter one of the time intervals is smaller than a threshold, or when the arrhythmia detection result for each of the electrocardiogram signal segments in the sequence of electrocardiogram signal segments corresponding to the shorter one of the time intervals is negative, determine the arrhythmia detection result for the electrocardiogram signal segment in the sequence of electrocardiogram signal segments corresponding to the shorter one of the time intervals to be negative.

In some embodiments of the present disclosure, the processor is further configured to execute the computer-executable instructions to, for each electrocardiogram signal segment in the sequence of electrocardiogram signal segments corresponding to the shorter one of the time intervals in the respective two sequences of electrocardiogram signal segments corresponding to each two adjacent time intervals, when the arrhythmia probabilities or arrhythmia detection results for at least two respective electrocardiogram signal segments in the sequence of electrocardiogram signal segments corresponding to the longer one of the time intervals comprising the electrocardiogram signal segment in the sequence of electrocardiogram signal segments corresponding to the shorter one of the time intervals are inconsistent with each other, determine the arrhythmia detection result for the electrocardiogram signal segment in the sequence of electrocardiogram signal segments corresponding to the shorter one of the time intervals in accordance with the arrhythmia probability for the electrocardiogram signal segment in the sequence of electrocardiogram signal segments corresponding to the shorter one of the time intervals.

In some embodiments of the present disclosure, the group of time intervals comprises a first time interval and a second time interval, a length of the second time interval is twice of a length of the first time interval, the first time interval corresponds to a first sequence of electrocardiogram signal segments, and the second time interval corresponds to a second sequence of electrocardiogram signal segments. The processor is further configured to execute the computer-executable instructions to, for the first sequence of electrocardiogram signal segments and the second sequence of electrocardiogram signal segments, determine the arrhythmia detection result for each electrocardiogram signal segment in the first sequence of electrocardiogram signal segments in accordance with the arrhythmia probability for the electrocardiogram signal segment in the first sequence of electrocardiogram signal segments, and the arrhythmia probability for the respective electrocardiogram signal segment in the second sequence of electrocardiogram signal segments, wherein the respective electrocardiogram signal segment in the second sequence of electrocardiogram signal segments comprises the electrocardiogram signal segment in the first sequence of electrocardiogram signal segments.

In yet another aspect, the present disclosure provides in some embodiments an arrhythmia detection system, including an electrocardiogram signal acquisition circuit configured to acquire an electrocardiogram signal, and the above-mentioned arrhythmia detection device being in communication with the electrocardiogram signal acquisition circuit and configured to receive and process the electrocardiogram signal from the electrocardiogram signal acquisition circuit.

In still yet another aspect, the present disclosure provides in some embodiments an arrhythmia detection system, including: an electrocardiogram signal acquisition circuit configured to acquire an electrocardiogram signal; a setting circuit configured to set a group of time intervals with gradually increasing lengths, wherein for each two adjacent time intervals in the group of time intervals, a length of a latter one time interval is an integral multiple of a length of a former one time interval; a division circuit configured to equally divide the electrocardiogram signal acquired by the electrocardiogram signal acquisition circuit through each of the time intervals to acquire respective sequences of electrocardiogram signal segments, wherein each of the time intervals in the group of time intervals corresponds to a respective one sequence of electrocardiogram signal segments; a first memory configured to store therein the sequences of electrocardiogram signal segments acquired by the division circuit; a determination circuit configured to read each of the sequences of electrocardiogram signal segments from the first memory, and determine an arrhythmia probability for each electrocardiogram signal segment in each of the sequences of electrocardiogram signal segments; a second memory configured to store therein the arrhythmia probability for each electrocardiogram signal segment in the each sequence of electrocardiogram signal segments determined by the determination circuit; and a detection circuit configured to read each of the sequences of electrocardiogram signal segments from the first memory, read the arrhythmia probability for each electrocardiogram signal segment in each of the sequences of electrocardiogram signal segments from the second memory, and for respective two sequences of electrocardiogram signal segments corresponding to each two adjacent time intervals, determine an arrhythmia detection result for each electrocardiogram signal segment in the sequence of electrocardiogram signal segments corresponding to a shorter one of the time intervals in accordance with the arrhythmia probability for the electrocardiogram signal segment in the sequence of electrocardiogram signal segments corresponding to the shorter one of the time intervals, and the arrhythmia probability or an arrhythmia detection result for a respective electrocardiogram signal segment in the sequence of electrocardiogram signal segments corresponding to a longer one of the time intervals, wherein the respective electrocardiogram signal segment in the sequence of electrocardiogram signal segments corresponding to a longer one of the time intervals comprises the electrocardiogram signal segment in the sequence of electrocardiogram signal segments corresponding to the shorter one of the time intervals.

It should be appreciated that, the general description above and the following detailed description are for illustrative purposes only, but shall not be construed as limiting the scope of the present disclosure.

An overview of the implementations or examples, rather than full disclosure of the entire scope or all features of the present disclosure, is provided hereinabove.

### BRIEF DESCRIPTION OF THE DRAWINGS

In order to illustrate the technical solutions of the present disclosure in a clearer manner, the drawings desired for the present disclosure will be described hereinafter briefly. Obviously, the following drawings merely relate to some embodiments of the present disclosure, but shall not be construed as limiting the scope of the present disclosure.

FIG. 1 is a schematic view showing an auricular fibrillation waveform within a time period of 6 seconds;

FIG. 2 is a block diagram of an arrhythmia detection system according to some embodiments of the present disclosure;

FIG. 3 is a flow chart of an arrhythmia detection method executed by a processor according to some embodiments of the present disclosure;

FIG. 4(a) is a schematic view showing the division of an electrocardiogram signal through the arrhythmia detection method implemented by a processor according to some embodiments of the present disclosure;

FIG. 4(b) is a flow chart of the determination of an arrhythmia label for each electrocardiogram signal segment in a sequence of electrocardiogram signal segments through the arrhythmia detection method implemented by the processor according to some embodiments of the present disclosure;

FIG. 5(a) is another schematic view showing the division of the electrocardiogram signal through the arrhythmia detection method implemented by the processor according to some embodiments of the present disclosure;

FIG. 5(b) is another flow chart of the determination of the arrhythmia label for each electrocardiogram signal segment in the sequence of electrocardiogram signal segments through the arrhythmia detection method implemented by the processor according to some embodiments of the present disclosure; and

FIG. 6 is another block diagram of the arrhythmia detection system according to some embodiments of the present disclosure.

### DETAILED DESCRIPTION OF THE EMBODIMENTS

In order to make the objects, the technical solutions and the advantages of the present disclosure more apparent, the present disclosure will be described hereinafter in a clear and complete manner in conjunction with the drawings and embodiments. Obviously, the following embodiments merely relate to a part of, rather than all of, the embodiments of the present disclosure, and based on these embodiments, a person skilled in the art may, without any creative effort, obtain the other embodiments, which also fall within the scope of the present disclosure.

Unless otherwise defined, any technical or scientific term used herein shall have the common meaning understood by a person of ordinary skills. Such words as “first” and “second” used in the specification and claims are merely used to differentiate different components rather than to represent any order, number or importance. Similarly, such words as “one” or “one of” are merely used to represent the existence of at least one member, rather than to limit the number thereof. Such words as “including” or “comprising” are merely used to represent an object or element presented before the word contains objects or elements or equivalences presented after the word, and the object or element presented before the word may further contains other objects or elements. Such words as “connect/connected to” or “couple/coupled to” may include electrical connection, direct or indirect, rather than to be limited to physical or mechanical connection. Such words as “on”, “under”, “left” and “right” are merely used to represent relative position relationship, and when an absolute position of the object is changed, the relative position relationship will be changed too.

For clarification, any known functions and components have been omitted in the following description.

A current electrocardio monitoring and detection system has such defects as insufficient positioning accuracy, omission and false detection. In addition, in the related art, it is necessary to divide (truncate) an electrocardiogram signal into various electrocardiogram signal segments. It is found that, the positioning accuracy and a detection rate of arrhythmia is associated with a length of each electrocardiogram signal segment (i.e., a truncated length of the electrocardiogram signal).

For example, for each electrocardiogram signal segment in a training data set of electrocardiogram signals, usually a detection result of at least one type of arrhythmia for the electrocardiogram signal segment may be determined to be positive in the case that an occurrence time of the at least one type of arrhythmia in the electrocardiogram signal segment exceeds ½ of a length of the electrocardiogram signal segment, and then a label for the at least one type of arrhythmia maybe set for the electrocardiogram signal segment. For example, when an electrocardiogram signal segment has a length of 10 seconds and an occurrence time of auricular fibrillation is more than 5 seconds, a label for the auricular fibrillation may be set for the electrocardiogram signal segment (i.e., the arrhythmia detection result for the electrocardiogram signal segment is that there exists the auricular fibrillation), and the labels for the other types of arrhythmias may be set (i.e., the detection result may be determined) in a similar way. Hence, when the electrocardiogram signal is divided into a plurality of electrocardiogram signal segments each having a length of L, the positioning accuracy of the arrhythmia detection may be L/2. At this time, merely the arrhythmia whose occurrence time is greater than or equal to L/2 may be detected, and the arrhythmia whose occurrence time is smaller than L/2 may not be detected.

However, the length of each electrocardiogram signal segment shall not be too small while the high positioning accuracy is achieved. This is because, the larger the length L, the more the information about an electrocardio waveform. Especially for the auricular fibrillation or ventricular fibrillation, its main feature lies in the irregularity within an R-R wave interval (i.e., a period of R wave, a time length of a heartbeat). This irregularity may be reflected in a more accurate manner when a longer electrocardiogram signal segment is provided, and thereby the arrhythmia detection rate may increase.

FIG. 1 shows an auricular fibrillation waveform within a time period of 6 seconds. When the length L is too small, e.g. 2 seconds, the R waves are distributed evenly within the first time period of 2 seconds. At this time, the electrocardiogram signal may be falsely determined as a normal one. Two R waves are distributed within the second time period of 2 seconds and three R waves are distributed within the third time period of 2 seconds, and a heart rate is relatively low. At this time, it is difficult to determine the detection result. In other words, it is difficult to determine the irregularity within the R-R interval when the length of the electrocardiogram signal segment is 2 seconds, but it is easy to determine the irregularity in accordance with the entire electrocardio waveform within the time period of 6 seconds. Hence, the length of each electrocardiogram signal segment shall not be too small, so as to prevent the occurrence of omission and false detection.

An object of the present disclosure is to provide an arrhythmia detection device and an arrhythmia detection system, so as to equally divide an electrocardiogram signal into a plurality of sequences of electrocardiogram signal segments through different time intervals in such a manner that each sequence corresponds to one time interval, and enable a sequence of electrocardiogram signal segments corresponding to a longer time interval (for ensuring that more information is taken into consideration) and a sequence of electrocardiogram signal segments corresponding to a shorter time interval (for ensuring the positioning accuracy) to form a sequence pair, thereby to make a compromise between the arrhythmia detection rate and the positioning accuracy with a clinically acceptable computational time.

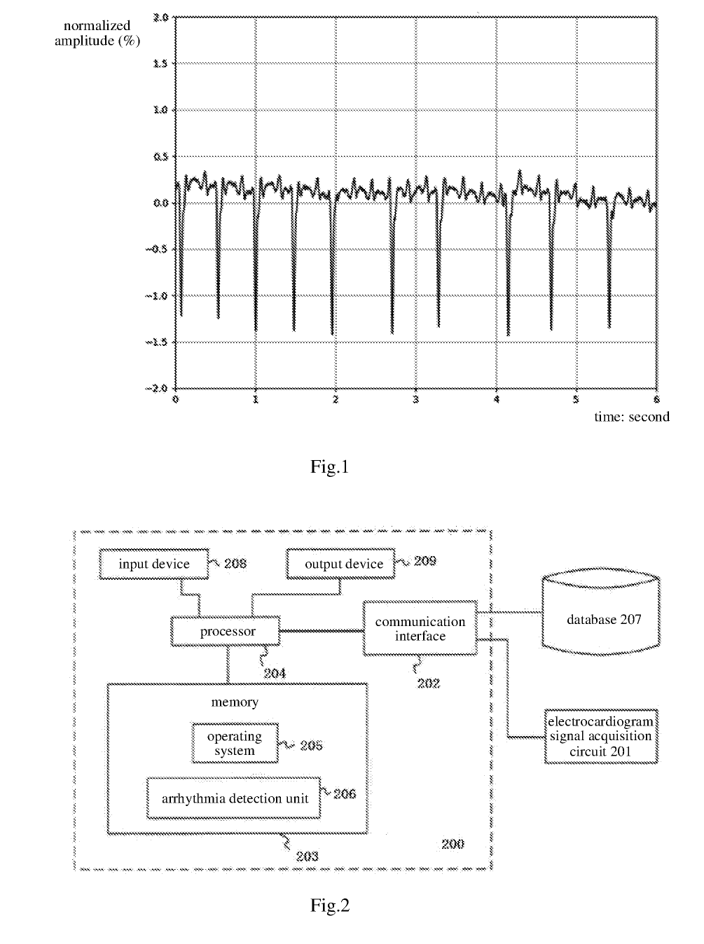

The present disclosure provides in some embodiments an arrhythmia detection system including an arrhythmia detection device 200, a database 207 and an electrocardiogram signal acquisition circuit 201. As shown in FIG. 2, the arrhythmia detection device 200 includes a memory 203 stored therein a computer-executable instruction, and a processor 204 configured to execute the computer-executable instruction, so as to perform the following operations.

The processor 204 may set a group of time intervals I₁, I₂, . . . , Iₙ with gradually increasing lengths (where n represents the quantity of the time intervals). A latter time interval Iₜ₊₁ (a longer time interval, where t is any integer within the range of 1 to n−1) of two adjacent time intervals may have a length as an integral multiple of a former time interval Iₜ (a shorter time interval).

The processor 204 may equally divide the electrocardiogram signal through each of the time intervals I₁, I₂, . . . , Iₙ, so as to acquire respective sequences S₁, S₂, . . . , Sₙ of electrocardiogram signal segments. In other words, the electrocardiogram signal may be equally divided through the time interval I₁, and a length of each electrocardiogram signal segment in the sequence S₁ may be I₁; the electrocardiogram signal may be equally divided through the time interval I₂, and a length of each electrocardiogram signal segment in the sequence S₂ may be I₂; . . . ; and the electrocardiogram signal may be equally divided through the time interval Iₙ, and a length of each electrocardiogram signal segment in the sequence Sₙ may be Iₙ. As a result, the sequences S₁ to Sₙ are obtained with respect to one electrocardiogram signal. Each time interval Iₜ may correspond to one respective sequence Sₜ of electrocardiogram signal segments, and a length of each electrocardiogram signal segment in the sequence Sₜ may be Iₜ.

Each sequence Sₜ may consist of a plurality of electrocardiogram signal segments each having a length of Iₜ. With respect to the individual sequence Sₜ, it may determine an arrhythmia probability for each electrocardiogram signal segment in the sequence Sₜ in various ways, e.g., through a detection model. The detection model may be established and trained with respect to the respective time interval, so as to determine the arrhythmia probability for each electrocardiogram signal segment corresponding to the time interval, which will be described hereinafter. For example, in order to determine the arrhythmia probability for each electrocardiogram signal segment corresponding to each of the time intervals I₁, I₂, . . . , Iₙ, n detection models (i.e., a first detection model to an n^{th} detection model) may be provided. For the sequence Sₜ, a t^{th} detection model corresponding to the time interval Iₜ may be adopted to determine the arrhythmia probability for each electrocardiogram signal segment. When the sequence Sₜ is divided into j electrocardiogram signal segments, the arrhythmia probabilities for the j electrocardiogram signal segments may be pt₁, pt₂, . . . , and ptⱼ.

In two adjacent time intervals Iₜ and Iₜ₊₁, the time interval Iₜ₊₁ is longer than the time interval Iₜ. For two sequences Sₜ and Sₜ₊₁ of electrocardiogram signal segments corresponding to the two adjacent time intervals Iₜ and Iₜ₊₁ (t may be any integer within the range of 1 to n−1), it may determine an arrhythmia detection result for each electrocardiogram signal segment in the sequence Sₜ of electrocardiogram signal segments corresponding to the time interval Iₜ in accordance with the arrhythmia probabilities pt₁, pt₂, . . . , and pt₃ for the electrocardiogram signal segments in the sequence Sₜ of electrocardiogram signal segments corresponding to the time interval Iₜ, and, as a dominant parameter, the arrhythmia probability or an arrhythmia detection result for each electrocardiogram signal segment in the sequence Sₜ₊₁ of electrocardiogram signal segments corresponding to the time interval Iₜ₊₁ and including the respective electrocardiogram signal segments in the sequence Sₜ of electrocardiogram signal segments corresponding to the time interval Iₜ.

In the context, when a certain electrocardiogram signal segment “includes” a plurality of respective electrocardiogram signal segments, it means that parts of the plurality of respective electrocardiogram signal segments form the electrocardiogram signal segment, or a portion of a combined segment consisting of the plurality of respective electrocardiogram signal segments is just the electrocardiogram signal segment. For example, when a 4-second portion of an electrocardiogram signal segment having a length of 8 seconds is just an electrocardiogram signal segment having a length of 4 seconds, the electrocardiogram signal segment having the length of 8 seconds may include the respective electrocardiogram signal segments (or one of the respective signal segments) each having the length of 4 seconds. For another example, when a 4-second portion of each of two electrocardiogram signal segments each having a length of 8 seconds is just a half of another electrocardiogram signal segment having a length of 8 seconds (i.e., the two 4-second portions together form the other electrocardiogram signal segment having the length of 8 seconds), the two electrocardiogram signal segments each having the length of 8 seconds may include the respective electrocardiogram signal segment having the length of 8 seconds.

In addition, the expression “as a dominant parameter” refers to that the parameter is provided with a larger weight factor or larger logic rule for the arrhythmia detection result, which will be described hereinafter in more details.

In some embodiments of the present disclosure, for the two adjacent time intervals, a start position of each electrocardiogram signal segment in the sequence Sₜ₊₁ of electrocardiogram signal segments corresponding to the time interval Iₜ₊₁ may be aligned with a start position of a respective electrocardiogram signal segment in the sequence Sₜ of electrocardiogram signal segments corresponding to the time interval Iₜ. In this way, an entirety of each electrocardiogram signal segment corresponding to the time interval Iₜ may be included in one electrocardiogram signal segment corresponding to the time interval Iₜ₊₁, rather than included in several electrocardiogram signal segments corresponding to the time interval Iₜ₊₁. The electrocardiogram signal segment corresponding to the time interval Iₜ₊₁ includes the entire electrocardiogram signal segment corresponding to the time interval Iₜ, so it may include more information. When the arrhythmia probability or arrhythmia detection result for each electrocardiogram signal segment in the sequence Sₜ₊₁ is taken as the dominant parameter for the arrhythmia detection result for the electrocardiogram signal segment in the sequence Sₜ, it is able to further improve an arrhythmia detection rate.

For example, an arrhythmia detection result for a certain electrocardiogram signal segment Seg_{t,m} (wherein m is a natural number smaller than or equal to j) in the sequence Sₜ corresponding to the time interval Iₜ may be determined in accordance with an arrhythmia probability for the electrocardiogram signal segment Seg_{t,m}, and as a dominant parameter, an arrhythmia probability or an arrhythmia detection result for an electrocardiogram signal segment Seg_{t+1,f} (f is a natural number smaller than or equal to the total quantity of the electrocardiogram signal segments) of all the electrocardiogram signal segments Seg_{t+1,k} of the sequence Sₜ₊₁ (k is an integer within the range of 1 to the quantity of the electrocardiogram signal segments in the sequence Sₜ₊₁) corresponding to the time interval Iₜ₊₁ including the electrocardiogram signal segment Seg_{t,m}.

In some embodiments of the present disclosure, during the determination of the detection result, the arrhythmia detection result for each of respective electrocardiogram signal segments corresponding to the time interval Iₜ₋₁ may be determined in accordance with, as a dominant parameter, the arrhythmia probability for each electrocardiogram signal segment corresponding to the time interval Iₜ (t is any integer within the range from 1 to n) and including the respective electrocardiogram signal segments corresponding to the time interval Iₜ₋₁. In this way, depending on the positioning accuracy, e.g., a half of the length of Iₜ₋₁, a user may determine the arrhythmia detection result for each electrocardiogram signal segment corresponding to the time interval Iₜ₋₁ while taking the information included in the electrocardiogram signal segments corresponding to the longer time interval Iₜ into consideration, so as to increase the arrhythmia detection rate with the given positioning accuracy.

In some embodiments of the present disclosure, the above-mentioned detection result determination may be performed on each two adjacent time intervals Iₜ and Iₜ₊₁, i.e., on each two adjacent time intervals of the groups I₁, I₂, . . . , L with gradually increasing lengths starting from a maximum time interval Iₙ, until the arrhythmia detection result for each electrocardiogram signal segment Seg₁ in the sequence S₁ corresponding to the shortest time interval I₁ has been determined. To be specific, during the determination of the detection result for each two adjacent time intervals, the arrhythmia probability, rather than the arrhythmia detection result, for each electrocardiogram signal segment corresponding to the maximum time interval Iₙ may play a dominant role in the determination of the arrhythmia detection result for each electrocardiogram signal segment corresponding to the time interval Iₙ₋₁ included in the corresponding electrocardiogram signal segment corresponding to the time interval Iₙ, and the arrhythmia detection result, rather than the arrhythmia probability, for each electrocardiogram signal segment corresponding to the time interval Iₜ (t is any integer within the range of 2 to n−1) may play a dominant role in the determination of the arrhythmia detection result for each electrocardiogram signal segment corresponding to the time interval Iₜ₋₁ included in the corresponding electrocardiogram signal segment corresponding to the time interval Iₜ. As a result, it is able for the arrhythmia detection system to achieve the positioning accuracy which is half of the length of the electrocardiogram signal segment in the sequence S₁ corresponding to the shortest time interval I₁ while taking the information included in each electrocardiogram signal segment in the sequence Sₙ corresponding to the maximum time interval Iₙ into consideration and taking the arrhythmia probability or the arrhythmia detection result for the electrocardiogram signal segment in the sequence Sₙ as a dominant parameter, thereby to increase the arrhythmia detection rate.

The arrhythmia detection result may include a positive result and a negative result, which are adopted to represent whether the arrhythmias have been detected. In some embodiments of the present disclosure, the arrhythmia detection result may include, but not limited to, a label indicating an arrhythmia condition. For example, the label may be “0” for indicating that no arrhythmia has been detected, or “1” for indicating that the arrhythmia has been detected. In addition, the label may also be adopted to indicate the other arrhythmia condition, e.g., “the auricular fibrillation has been detected”, “no sinus arrhythmia has been detected”, or “an arrhythmia at a large therapeutic risk level has been detected”.

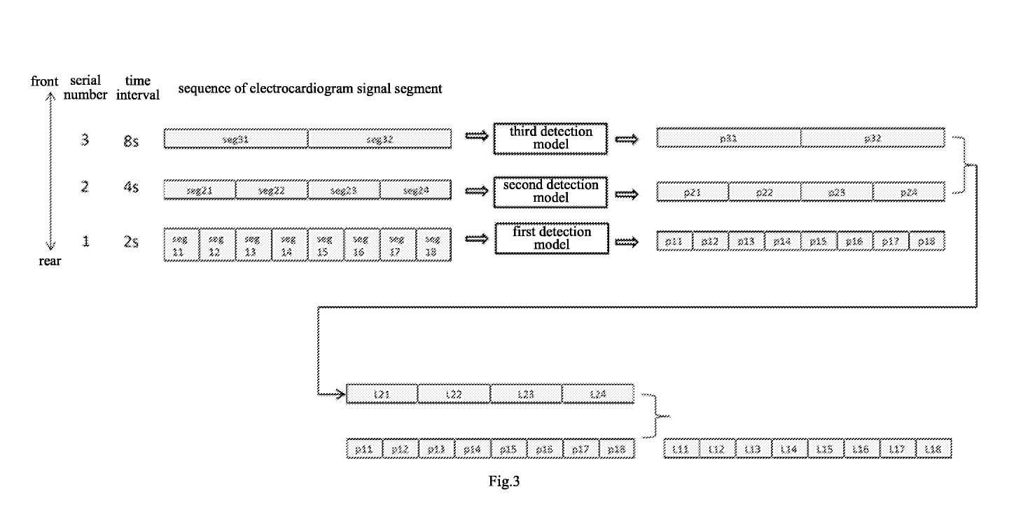

The following description will be given by taking the above labels “0” and “1” as an example in conjunction with FIG. 3. It should be appreciated that, in the following, an arrhythmia detection method executed by the processor 204 will be described when the arrhythmia detection results are shown by the labels “0” and “1”, and any other modifications may be acquired when the label is adopted to indicate the other arrhythmia detection results, which will not be particularly defined herein. In addition, when the arrhythmia is detected in accordance with the arrhythmia probability, a threshold of 0.5 may be adopted. However, the threshold may not be limited to 0.5, and any other values may also be adopted so as to acquire the other modifications, which will not be particularly defined herein.

As shown in FIG. 3, the acquired electrocardiogram signal may be divided into sequences S₃, S₂ and S₁ of electrocardiogram signal segments through a group of time intervals of 8 seconds (a maximum time interval), 4 seconds, and 2 seconds (a minimum time interval) respectively. In a front-and-rear direction in FIG. 3, for each two adjacent time intervals, a length of a former one (i.e., a longer time interval) may be twice of a length of a latter one (i.e., a shorter time interval), and a start position of each electrocardiogram signal segment in a former sequence (i.e., the sequence corresponding to the longer time interval) may be aligned with a start position of a corresponding electrocardiogram signal segment in a latter sequence (i.e., the sequence corresponding to the shorter time interval). For example, a start position of a first electrocardiogram signal segment in the sequence S₃ is aligned with a start position of a first electrocardiogram signal segment in the sequence S₂.

For each of the sequences S₁, S₂ and S₃, the arrhythmia probability for each electrocardiogram signal segment may be determined in various ways, e.g., through a detection model corresponding to the time interval (i.e., one of a first detection model, a second detection model and a third detection model). The detection model may be established and trained with respect to the time interval, so as to determine the arrhythmia probability for each electrocardiogram signal segment corresponding to the time interval. In some embodiments of the present disclosure, each detection model may be trained with respect to the electrocardiogram signal segments corresponding to the time interval, and then implemented through machine learning, e.g., implemented through any one of a perceptron model, a k-nearest neighbor model, a support vector machine model and a neural network model. In addition, apart from the time interval, each detection model may also be established and trained with respect to various arrhythmias, for example, with respect to the arrhythmias depending on the body parts, e.g., one or more of sinus arrhythmia, atrial arrhythmia, junctional arrhythmia occurring at an atrioventricular junction and ventricular arrhythmia, or with respect to the arrhythmias having a large risk level and requiring treatment, e.g., one or more of auricular fibrillation, auricular flutter, supraventricular tachycardia, auricular tachycardia with block, frequent premature contraction, premature ventricular contraction of a special type, pre-excitation syndrome with tachycardia, two-degree Mohs II block, three-degree atrioventricular block and severe bradycardia. In this way, it is able to detect various arrhythmias, thereby to help a doctor to position a location where the arrhythmia occurs and select an appropriate treatment means. In addition, it is able to detect the arrhythmia at different risk levels, and help the doctor to find a disease to be handled urgently at an early stage, thereby to prevent the occurrence of a mortal danger due to delayed treatment or prevent the occurrence of misallocation of medical resources for less severe diseases.

For the sequence S₃, the arrhythmia probabilities p31 and p32 for the electrocardiogram signal segments seg31 and seg32 may be determined through the third detection model. For the sequence S₂, the arrhythmia probabilities p21, p22, p23 and p24 for the electrocardiogram signal segments seg21, seg22, seg23 and seg24 may be determined through the second detection model. For the sequence S₁, the arrhythmia probabilities p11, p12, p13, p14, p15, p16, p17 and p18 for the electrocardiogram signal segments seg11, seg12, seg13, seg14, seg15, seg16, seg17 and seg18 may be determined through the third detection model.

As shown in FIG. 3, the following calculation may be performed progressively from the sequence S₃ backwardly (i.e., starting from the sequence corresponding to the maximum time interval). To be specific, for the sequences S₃ and S₂ corresponding to the time intervals of 8 seconds and 4 seconds respectively, the detection result may be determined as follows. The arrhythmia labels L21, L22, L23 and L24 for the electrocardiogram signal segments seg21, seg22, seg23 and seg24 in the sequence S₂ may be determined in accordance with the arrhythmia probabilities p21, p22, p23 and p24 for the electrocardiogram signal segments seg21, seg22, seg23 and seg24 in the sequence S₂ (corresponding to a shorter time interval), and as a dominant parameter, the arrhythmia probabilities p31, p31, p32 and p32 for the respective electrocardiogram signal segments seg31, seg31, seg32 and seg32 in the sequence S₃ (corresponding to the longer time interval) including the electrocardiogram signal segments seg21, seg22, seg23 and seg24 respectively. Next, for the sequences S₂ and S₁ corresponding to the adjacent time intervals of 4 seconds and 2 seconds respectively, the detection result may be determined as follows. The arrhythmia labels L11, L12, L13, L14, L15, L16, L17 and L18 for the electrocardiogram signal segments seg11, seg12, seg13, seg14, seg15, seg16, seg17 and seg18 in the sequence S₁ may be determined in accordance with the arrhythmia probabilities p11, p12, p13, p14, p15, p16, p17 and p18 for the electrocardiogram signal segments seg11, seg12, seg13, seg14, seg15, seg16, seg17 and seg18 in the sequence S₁, and as a dominant parameter, the arrhythmia labels L21, L21, L22, L22, L23, L23, L24 and L24 for the respective electrocardiogram signal segments seg21, seg21, seg22, seg22, seg23, seg23, seg24 and seg24 in the sequence S₂ including the electrocardiogram signal segments seg11, seg12, seg13, seg14, seg15, seg16, seg17 and seg18 respectively. It should be appreciated that, when the arrhythmia label L=1, it means that the arrhythmia occurs for the electrocardiogram signal segment, and when the arrhythmia label L=0, it means that the electrocardiogram signal segment is a normal one.

In some embodiments of the present disclosure, for the sequences Sₜ and Sₜ₊₁ corresponding to two adjacent time intervals, the label may be determined in various ways. For example, weighted calculation may be performed on the arrhythmia probabilities pt1, pt2, . . . , ptj for the electrocardiogram signal segments in the sequence Sₜ corresponding to the shorter time interval and the arrhythmia probabilities for the respective electrocardiogram signal segments in the sequence Sₜ₊₁ corresponding to the longer time interval including the electrocardiogram signal segments in the sequence Sₜ, and a larger weight value may be applied to the latter, so as to enable the latter to play a dominant role. As a result, the weighted probabilities for the electrocardiogram signal segments in the sequence Sₜ corresponding to the shorter time interval. Then, the arrhythmia labels for the electrocardiogram signal segments in the sequence Sₜ corresponding to the shorter time interval may be determined in accordance with the weighted probabilities. For example, when the time interval corresponding to the sequence Sₜ₊₁, e.g., S₂ in FIG. 3, is not the maximum time interval, the weighted calculation may be performed on the arrhythmia probabilities p11, p12, p13, p14, p15, p16, p17 and p18 for the electrocardiogram signal segments seg11, seg12, seg13, seg14, seg15, seg16, seg17 and seg18 in a next sequence, i.e., the sequence S₁, and the arrhythmia probabilities represented by the arrhythmia labels L21, L21, L22, L22, L23, L23, L24 and L24 for the electrocardiogram signal segments seg21, seg21, seg22, seg22, seg23, seg23, seg24 and seg24 in the sequence S₂ including the electrocardiogram signal segments seg11, seg12, seg13, seg14, seg15, seg16, seg17 and seg18 respectively, so as to determine the arrhythmia labels L11, L12, L13, L14, L15, L16, L17 and L18 for the electrocardiogram signal segments seg11, seg12, seg13, seg14, seg15, seg16, seg17 and seg18 in the sequence S₁. In some embodiments of the present disclosure, the probability represented by each arrhythmia label may be acquired by modifying the arrhythmia probability corresponding to the arrhythmia label. For example, when p21 is smaller than a threshold (e.g., 0.5) and L21 is 1, p21 may be modified into p21′ which is greater than or equal to the threshold, so as to represent the probability. However, any other modification ways may also be adopted, so as to modify the arrhythmia probability to be consistent with the corresponding arrhythmia label.

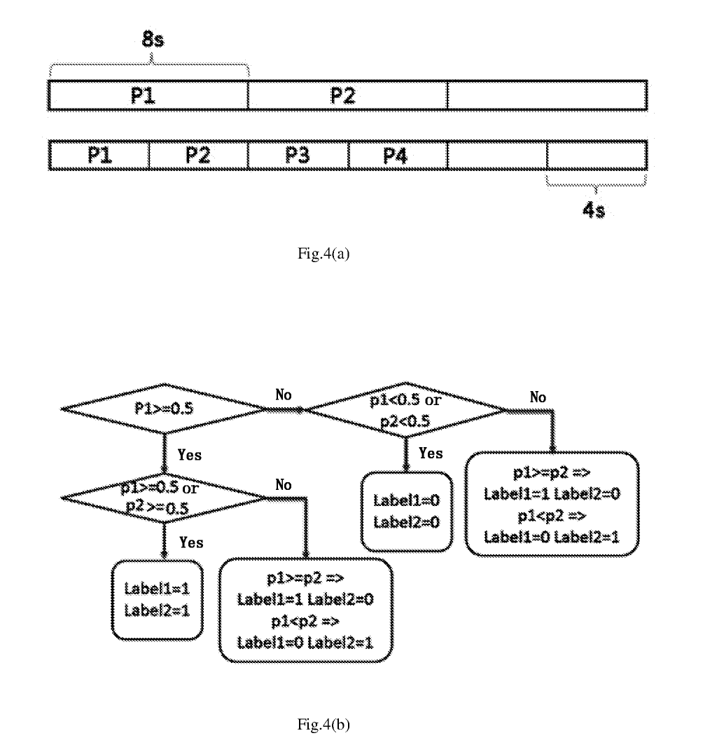

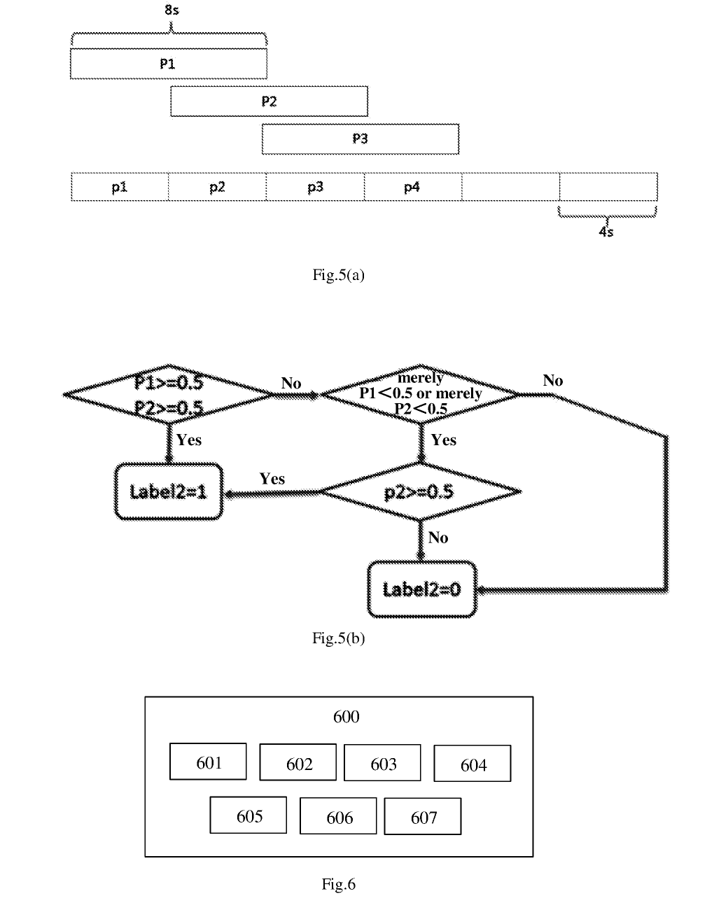

For example, when the weighted probability is greater than or equal to 0.5, the arrhythmia label L for the corresponding electrocardiogram signal segment may be 1, i.e., the arrhythmia has occurred. Otherwise, the arrhythmia label L for the corresponding electrocardiogram signal segment may be 0, i.e., the electrocardiogram signal segment is a normal one. However, the determination of the label may not be limited to that mentioned above, and more examples will be described hereinafter in conjunction with FIGS. 4(a), 4(b), 5(a) and 5(b).

The processor 204 may be in communication with memory 203, and configured to execute the computer-executable instruction stored in the memory 203. For example, the processor 204 may execute an operating system 205 stored in the memory 203, or the computer-executable instruction for the arrhythmia detection in the arrhythmia detection unit 206 stored in the memory 203. In some embodiments of the present disclosure, the computer-executable instruction for the arrhythmia detection may be stored in the memory 203 in the form of an application (i.e., the arrhythmia detection unit 206). However, it should be appreciated that, the arrhythmia detection unit is provided on the basis of its function, and it may also be integrated with an electrocardiogram signal detection and analysis module.

The processor 204 may be a processing unit, e.g., a general-purpose processing unit, including one or more of a microprocessor, a Central Processing Unit (CPU), a Graphical Processing Unit (GPU), and an Accelerated Processing Unit (APU). To be specific, the processor 204 may be a Complex Instruction Set Computing (CISC) microprocessor, Reduced Instruction Set Computing (RISC) microprocessor, a Very Long Instruction Word (VLIW) microprocessor, a processor for executing other instruction sets, or a processor for executing a combination of instruction sets. In addition, the processor 204 may also include one or more dedicated processing units, e.g., an Application Specific Integrated Circuit (ASIC), a Field Programmable Gate Array (FPGA), a Digital Signal Processor (DSP) or a System On Chip (SOC). In some embodiments of the present disclosure, the processor 204 may be a dedicated processor rather than a general-purpose processor. The processor 204 may include one or more known processing units, e.g., Pentium™, Core™, Xeon™ or Itanium® microprocessors from Intel™, or Turion™, Athlon™, Sempron™, Opteron™, FX™ or Phenom™ microprocessors from AMD™, or processors from Sun Microsystems. The processor 204 may further include an accelerated processing unit, e.g., a desk-type A-4(6,8) accelerated processing unit from AMD™, or an Xeon Phi™ accelerated processing unit from Intel™. The type and the quantity of the processors will not be particularly defined herein, as long as the processor may be configured to identify, analyze, maintain, generate and/or provide a large quantity of electrocardiogram signal data, or process the electrocardiogram signal data or any other appropriate data in the embodiments of the present disclosure. In addition, more than one processor may be provided, e.g., a processor with multiple cores or a plurality of processors each with multiple cores. The processor 204 may execute a sequence of computer program instructions stored in the memory 203, so as to implement the operations, processings and methods in the embodiments of the present disclosure.

For example, the memory 203 may include one computer program product or a combination of computer program products. The computer program product may include various computer-readable storage mediums, e.g., a volatile memory and/or a nonvolatile memory. The volatile memory may include a Random Access Memory (RAM) and/or a cache. The nonvolatile memory may include a Read Only Memory (ROM), a hard disc, an Erasable Programmable ROM (EPROM), a Compact Disc-ROM (CD-ROM), a Universal Serial Bus (USB) memory, or a flask memory.

Various applications and data, e.g., sample data about the electrocardiogram signals, training data, various data adopted and/or generated by the applications, or algorithms for the various detection models, may be further stored in the computer-readable storage medium.

In some embodiments of the present disclosure, the arrhythmia detection device 200 may further include a communication interface 202 configured to receive the electrocardiogram signal from the electrocardiogram signal acquisition circuit 201.

In some embodiments of the present disclosure, the arrhythmia detection device 200 may further include an output unit 209 configured to output the detection result. For example, the output unit 209 may be a display, a printer or a projector, so as to display the detection result and/or the corresponding electrocardiogram signal waveform. The doctor may view the detection result and/or the electrocardiogram signal waveform through the display, a printed file or a screen of the projector.

In some embodiments of the present disclosure, the arrhythmia detection device 200 may further include an input device 208, e.g., a keyboard, a mouse, a remote controller or a touch panel having a touch function, which is configured to receive an instruction from an external computer device or a user (e.g., the doctor). The doctor may interact with the arrhythmia detection device 200 via the input device 208. For example, the doctor may set, via the input device 208, the time interval for the electrocardiogram signal segment, set the quantity of sequences (layers) of the electrocardiogram signal segments, or select different detection models from a menu, or the like.

The communication interface 202 may include any mechanism coupled to any one of a hardwired medium, a radio medium and an optical medium so as to communicate with another device, e.g., a memory bus interface, a processor bus interface, an Internet connection or a magnetic disc controller. Through providing configuration parameters and/or transmitting a signal, the communication interface 202 may be configured to provide a data signal describing software content. The communication interface 202 may be accessed through transmitting thereto one or more commands or signals.

In some embodiments of the present disclosure, the arrhythmia detection device 200 may be connected to the database 207 via the communication interface 202. The plurality of electrocardiogram signals acquired by the electrocardiogram signal acquisition circuit 201 may be stored in the database 207, so that the arrhythmia detection device 200 may call the electrocardiogram signals. In addition, the detection result from the arrhythmia direction unit 206 and the corresponding electrocardiogram signals may be transmitted via the communication interface 202, and stored in, the database 207, so that the arrhythmia detection device 200 may call them as the training data set or the other user, e.g., the doctor, may call them for the diagnosis of a disease of cardiovascular system. The database 207 may include an Oracle™ database, Sybase™ database or another relational database, or a non-relational database such as Hadoop sequence file, HBase or Cassandra.

In some embodiments of the present disclosure, the arrhythmia detection device 200 and the electrocardiogram signal acquisition circuit 201 may be integrated as an electrocardiogram monitor.

In some embodiments of the present disclosure, the arrhythmia detection device 200 and the electrocardiogram signal acquisition circuit 201 may also form an arrhythmia detection system. The arrhythmia detection device 200 may be in communicating connection with the electrocardiogram signal acquisition circuit 201 via the communication interface 202, so as to receive the electrocardiogram signals from the electrocardiogram signal acquisition circuit.

The arrhythmia detection method executed by the processor 204 will be described hereinafter in more details in conjunction with the embodiments.

FIGS. 4(a) and 4(b) show the arrhythmia direction method executed by the processor in some embodiments of the present disclosure. Two time intervals are provided, i.e., a first time interval of 4 seconds and a second time interval of 8 seconds, and the second time interval has a length twice of a length of the first time interval.

As shown in FIG. 4(a), the electrocardiogram signal may be divided into two sequences of electrocardiogram signal segments through the time intervals of 8 seconds and 4 seconds. A first sequence may correspond to the first time interval 4 seconds, and a second sequence may correspond to the second time interval 8 seconds.

The arrhythmia probabilities for the first two electrocardiogram signal segments in the second sequence calculated through the second detection model may be P1 and P2 respectively, and the arrhythmia probabilities for the first four electrocardiogram signal segments in the first sequence calculated through the first detection model may be p1, p2, p3 and p4. It should be appreciated that, the electrocardiogram signal segments in each sequence may be consecutive, i.e., these electrocardiogram signal segments may not be spaced apart from each other, neither overlap each other, and instead, they are connected to each other in an end-to-end manner. In some embodiments of the present disclosure, a start position of a first electrocardiogram signal segment in the second sequence may be aligned with a start position of a first electrocardiogram signal segment in the first sequence, and the first electrocardiogram signal segment in the second sequence may include the first electrocardiogram signal segment and a second electrocardiogram signal segment in the first sequence. A start position of a second electrocardiogram signal segment in the second sequence may be aligned with a start position of a third electrocardiogram signal segment in the first sequence, and the second electrocardiogram signal segment in the second sequence may include the third electrocardiogram signal segment and a fourth electrocardiogram signal segment in the first sequence.

There are merely the first and second sequences, so the detection result determination may be performed merely with respect to the first sequence and the second sequence. As shown in FIG. 4(b), the following description will be given by taking the determination of the arrhythmia labels Label1 and Label2 for the first two electrocardiogram signal segments in the first sequence as an example. In the following, the threshold may be 0.5. It should be appreciated that the threshold may be set as another value.

When the arrhythmia probability P1 for the first electrocardiogram signal segment in the second sequence corresponding to the longer time interval of 8 seconds is greater than or equal to 0.5, Label1 and Label2 for the first and second electrocardiogram signal segments in the first sequence corresponding to the shorter time interval of 4 seconds included in the first electrocardiogram signal segment in the first sequence 2 may each be 1 (i.e., the arrhythmia direction results may each be positive) as long as one of the arrhythmia probabilities p1 and p2 for the first and second electrocardiogram signal segments in the first sequence is greater than or equal to 0.5. When the arrhythmia probabilities p1 and p2 for the first and second electrocardiogram signal segments in the first sequence corresponding to the shorter time interval of 4 seconds are each smaller than 0.5, it means that different results are acquired by the first detection model and the second detection model with respect to the same electrocardiogram signal segment. At this time, the result from the second detection model corresponding to the time interval of 8 seconds (i.e., the arrhythmia probability P1 for the first electrocardiogram signal segment in the second sequence corresponding to the longer time interval of 8 seconds is greater than or equal to 0.5) may be taken as a dominant parameter, so as to determine that one of Label1 and Label 2 for the first and second electrocardiogram signal segments in the first sequence is still 1 (i.e., the arrhythmia detection result is positive). In this way, the arrhythmia probability P1 for the first electrocardiogram signal segment in the second sequence corresponding to the longer time interval of 8 seconds (P1 is greater than or equal to 0.5) may play a dominant role in the detection result determination. For example, when p1 is greater than or equal to p2, Label1 may be 1 while Label2 may be 0, and otherwise Label1 may be 0 while Label2 may be 1.

In another possible embodiment of the present disclosure, when the first electrocardiogram signal segment in the second sequence corresponding to the longer time interval of 8 seconds includes the first to the m^{th} electrocardiogram signal segments in the first sequence, the arrhythmia probability P1 for the first electrocardiogram signal segment in the second sequence is greater than or equal to 0.5 and the arrhythmia probabilities p1 to pm for the first to the m^{th} electrocardiogram signal segments in the first sequence are each smaller than 0.5, the arrhythmia label for the electrocardiogram signal segment corresponding to the maximum one of p1 to pm may be 1. In another possible embodiment of the present disclosure, the arrhythmia labels for some electrocardiogram signal segments corresponding to some of p1 to pm having relatively large values (e.g., a half of p1 to pm, or a predetermined quantity of p1 to pm, having relatively large values), rather than all the electrocardiogram signal segments corresponding to p1 to pm, may each be 1.

When the arrhythmia probability P1 for the first electrocardiogram signal segment in the second sequence corresponding to the longer time interval of 8 seconds is smaller than 0.5, Label1 and Label2 for the first and second electrocardiogram signal segments in the first sequence corresponding to the shorter time interval of 4 seconds included in the first electrocardiogram signal segment in the first sequence 2 may each be 0 (i.e., the arrhythmia direction results may each be negative) as long as one of the arrhythmia probabilities p1 and p2 for the first and second electrocardiogram signal segments in the first sequence is smaller than 0.5. When p1 and p2 are each greater than or equal to 0.5, one of Label1 and Label2 may be 0 (i.e., the arrhythmia direction results may each be negative). In this way, the arrhythmia probability P1 for the first electrocardiogram signal segment in the second sequence corresponding to the longer time interval of 8 seconds (P1 is smaller than 0.5) may play a dominant role in the detection result determination. For example, when p1 is greater than or equal to p2, Label1 may be 1 while Label2 may be 0, and otherwise Label1 may be 0 while Label2 may be 1.

The above description is given by taking two sequences and two detection models as an example. It should be appreciated that an additional detection model, e.g., the detection model corresponding to the time interval of 2 seconds as shown in FIG. 3, may also be provided in accordance with the practical need, so as to increase the positioning accuracy to 2 seconds. In addition, an additional detection model corresponding to a time interval of 16 s may further be provided, so as to provide more waveform information, thereby to further increase the detection rate and the detection accuracy. In the case that more than two detection models are provided, the above-mentioned determination procedure may be adjusted, so as to determine the labels for the adjacent sequences. For example, the calculation may be performed for each two time intervals from the sequence corresponding to the maximum time interval. When the longer one of the adjacent time intervals is not the maximum time interval, the arrhythmia label for each electrocardiogram signal segment in the sequence corresponding to the longer time interval (e.g., the arrhythmia labels L21, L22, L23 and L24 for the electrocardiogram signal segments in the second sequence in FIG. 3) may be acquired at first. For each electrocardiogram signal segment in the sequence corresponding to the longer time interval, in the case that the arrhythmia label thereof is 1 and the arrhythmia probability for at least one electrocardiogram signal segment in the sequence corresponding to the shorter time interval included in the electrocardiogram signal segment in the sequence corresponding to the longer time interval is greater than 0.5, the arrhythmia labels for the electrocardiogram signal segments in the sequence corresponding to the shorter time interval may each be determined as 1. In the case that the arrhythmia label is 1 and the arrhythmia probability for each electrocardiogram signal segment in the sequence corresponding to the shorter time interval included in the electrocardiogram signal segment in the sequence corresponding to the longer time interval is smaller than 0.5, the arrhythmia label for at least one electrocardiogram signal segment, but not all the electrocardiogram signal segments, in the sequence corresponding to the shorter time interval may be determined to be 1. In this way, the arrhythmia label of 1 for the electrocardiogram signal segment in the sequence corresponding to the longer time interval may play a dominant role in the determination of the arrhythmia label for the electrocardiogram signal segments in the sequence corresponding to the shorter time interval included in the electrocardiogram signal segment in the sequence corresponding to the longer time interval. The arrhythmia probabilities for the electrocardiogram signal segments in the sequence corresponding to the shorter time interval may be compared with each other, so as to determine the electrocardiogram signal segment for which the arrhythmia label shall be 1. In some embodiments of the present disclosure, for each electrocardiogram signal segment in the sequence corresponding to the longer time interval, in the case that the arrhythmia label is 0 and the arrhythmia probability for at least one electrocardiogram signal segment in the sequence corresponding to the shorter time interval included in the electrocardiogram signal segment in the sequence corresponding to the longer time interval is smaller than 0.5, the arrhythmia label for each electrocardiogram signal segment in the sequence corresponding to the shorter time interval included in the electrocardiogram signal segment in the sequence corresponding to the longer time interval may be 0. In the case that the arrhythmia label is 0 and the arrhythmia probability for each electrocardiogram signal segment in the sequence corresponding to the shorter time interval included in the electrocardiogram signal segment in the sequence corresponding to the longer time interval is greater than 0.5, the arrhythmia label for at least one electrocardiogram signal segment, but not all the electrocardiogram signal segments, in the sequence corresponding to the shorter time interval included in the electrocardiogram signal segment in the sequence corresponding to the longer time interval may be 0. In this way, the arrhythmia label of 0 for the electrocardiogram signal segment in the sequence corresponding to the longer time interval may play a dominant role in the determination of the arrhythmia label for the electrocardiogram signal segments in the sequence corresponding to the shorter time interval included in the electrocardiogram signal segment in the sequence corresponding to the longer time interval. The arrhythmia probabilities for the electrocardiogram signal segments in the sequence corresponding to the shorter time interval may be compared with each other, so as to determine the electrocardiogram signal segment for which the arrhythmia label shall be 0.

FIGS. 5(a) and 5(b) also show the arrhythmia detection method executed by the processor in some embodiments of the present disclosure.

As shown in FIG. 5(a), the electrocardiogram signal may be divided into two sequences through the time intervals of 8 seconds and 4 seconds. A first sequence may correspond to a first time interval of 4 seconds, and a second sequence may correspond to a second time interval of 8 seconds. FIGS. 5(a) and 5(b) differ from FIGS. 4(a) and 4(b) in that the electrocardiogram signal segments in the second sequence overlap each other. The arrhythmia probabilities for the first three electrocardiogram signal segments in the second sequence calculated through the second detection model may be P1, P2 and P3, and the arrhythmia probabilities for the first four electrocardiogram signal segments in the first sequence calculated through the first detection model may be p1, p2, p3 and p4. When the electrocardiogram signal segments in the second sequence overlap each other, it is able to reuse information when insufficient information is contained in the electrocardiogram signal, thereby to increase the arrhythmia detection rate. As compared with the arrhythmia detection method in FIGS. 4(a) and 4(b), when the arrhythmia detection method in FIGS. 5(a) and 5(b) is applied to the electrocardiogram signal with a same length, the computational burden is relatively larger, but the arrhythmia detection rate may be further increased.

For example, an overlapping portion between the adjacent electrocardiogram signal segments in the second sequence may be just one electrocardiogram signal segment in the first sequence, as shown in FIG. 5(b). In some embodiments of the present disclosure, a start position of a first electrocardiogram signal segment in the second sequence may be aligned with a start position of a first electrocardiogram signal segment in the first sequence, and the first electrocardiogram signal segment in the second sequence may include the first electrocardiogram signal segment and a second electrocardiogram signal segment in the first sequence. A start position of a second electrocardiogram signal segment in the second sequence may be aligned with a start position of the second electrocardiogram signal segment in the first sequence, and the second electrocardiogram signal segment in the second sequence may include the second electrocardiogram signal segment and a third electrocardiogram signal segment in the first sequence. A start position of a third electrocardiogram signal segment in the second sequence may be aligned with a start position of the third electrocardiogram signal segment in the first sequence, and the third electrocardiogram signal segment in the second sequence may include the third electrocardiogram signal segment and a fourth electrocardiogram signal segment in the first sequence.

There are merely the first and second sequences, so the detection result determination may be performed merely with respect to the first sequence and the second sequence. As shown in FIG. 5(b), the following description will be given by taking the determination of the arrhythmia label Label2 for the second electrocardiogram signal segment in the first sequence (corresponding to the shorter time interval) as an example.

The overlapping portion between the first electrocardiogram signal segment and the second electrocardiogram signal segment in the second sequence may be just the second electrocardiogram signal segment in the first sequence. When the arrhythmia probabilities P1 and P2 for at least two electrocardiogram signal segments, e.g., the first and the second electrocardiogram signal segments, in the second sequence (corresponding to the longer time interval) including the second electrocardiogram signal segment in the first sequence (corresponding to the shorter time interval) are each greater than or equal to 0.5, Label2 for the second electrocardiogram signal segment in the first sequence may be 1 (i.e., the arrhythmia detection result may be positive). When the arrhythmia probabilities P1 and P2 for at least two electrocardiogram signal segments, e.g., the first and the second electrocardiogram signal segments, in the second sequence (corresponding to the longer time interval) including the second electrocardiogram signal segment in the first sequence (corresponding to the shorter time interval) are each smaller than 0.5, Label2 for the second electrocardiogram signal segment in the first sequence may be 0 (i.e., the arrhythmia detection result may be negative). In this way, the arrhythmia probabilities P1 and P2 for the first and the second electrocardiogram signal segments in the second sequence including the second electrocardiogram signal segment in the first sequence may play a dominant role in the determination of the label for the second electrocardiogram signal segment in the first sequence.

When P1 is different from P2, i.e., merely one of P1 and P2 is smaller than 0.5, the arrhythmia detection result for each electrocardiogram signal segment in the first sequence corresponding to the shorter time interval may be determined in accordance with the arrhythmia probability P2 of each electrocardiogram signal segment in the first sequence. For example, when p2 is greater than or equal to 0.5, Label2=1, and otherwise Label2=0.

The above description is given by taking two sequences and two detection models as an example. The longer time interval may be the maximum time interval, and the detection result determination may be performed with respect to two sequences corresponding to two adjacent time intervals. The arrhythmia probability, rather than the arrhythmia detection result, for the electrocardiogram signal segment in the sequence corresponding to the longer time interval may play a dominant role in the determination of the arrhythmia detection result for each electrocardiogram signal segment in the sequence corresponding to the shorter time interval included in the electrocardiogram signal segment in the sequence corresponding to the longer time interval.

Depending on the practical need, one or more additional detection models may also be provided. For example, an additional detection model corresponding to the time interval of 2 seconds may be provided, so as to increase the positioning accuracy of the detection to 2 seconds. For another example, an additional detection model corresponding to the time interval of 16 s may also be provided, so as to provide more waveform information, thereby to further increase the detection accuracy. In the case that more than two detection models and more than two sequences are provided, different from the situation where two sequences are provided, the detection result determination may be performed with respect to each two sequences corresponding to respective two adjacent time intervals, starting from the sequence corresponding to the maximum time interval. When the longer time interval of the two adjacent time intervals is the maximum time interval, the arrhythmia probability for the electrocardiogram signal segment in the sequence corresponding to the longer time interval may play a dominant role in the arrhythmia detection result for each electrocardiogram signal segment in the sequence corresponding to the shorter time interval included in the electrocardiogram signal segment in the sequence corresponding to the longer time interval. When the longer time interval of the two adjacent time intervals is not the maximum time interval, the arrhythmia detection result (for example, a tag indicating the arrhythmia condition) for the electrocardiogram signal segment in the sequence corresponding to the longer time interval may play a dominant role in the arrhythmia detection result for each electrocardiogram signal segment in the sequence corresponding to the shorter time interval included in the electrocardiogram signal segment in the sequence corresponding to the longer time interval. This modification may be implemented by a person skilled in the art on the basis of the above description.

In the case that more than two detection models are provided, the above-mentioned determination procedure may be adjusted, so as to determine the labels for the adjacent sequences. For example, when the sequence corresponding to the longer time interval in two adjacent sequences is not a top-layer sequence (i.e., not the sequence corresponding to the maximum time interval), the calculation may be performed progressively from the top-layer sequence, so as to acquire the arrhythmia label for each electrocardiogram signal segment in the sequence corresponding to the longer time interval. For each electrocardiogram signal segment in the sequence corresponding to the shorter time interval, in the case that the arrhythmia label for each electrocardiogram signal segment in the sequence corresponding to the longer time interval including the electrocardiogram signal segment in the sequence corresponding to the shorter time interval is 1, it may determine that the arrhythmia label for the respective electrocardiogram signal segment in the sequence corresponding to the shorter time interval may be 1. In the case that the arrhythmia label for each electrocardiogram signal segment in the sequence corresponding to the longer time interval including the electrocardiogram signal segment in the sequence corresponding to the shorter time interval is 0, it may determine that the arrhythmia label for the respective electrocardiogram signal segment in the sequence corresponding to the shorter time interval may be 0. In some embodiments of the present disclosure, for each electrocardiogram signal segment in the sequence corresponding to the shorter time interval, when the arrhythmia labels for the electrocardiogram signal segments in the sequence corresponding to the longer time interval including the electrocardiogram signal segment in the sequence corresponding to the shorter time interval are different from each other, the arrhythmia probabilities for the electrocardiogram signal segments in the sequence corresponding to the shorter time interval may be compared with each other, so as to determine the arrhythmia label for each electrocardiogram signal segment in the sequence corresponding to the shorter time interval. This modification may be implemented by a person skilled in the art on the basis of the above description.

Based on an electrocardiogram signal No. 8219 (including normal heartbeats for 28874 seconds and auricular fibrillation heartbeats for 7949 seconds) in an MIT-BIH auricular fibrillation database, the scheme in the embodiments of the present disclosure was tested through the first detection model corresponding to the time interval of 4 seconds, the second detection model corresponding to the time interval of 8 seconds, the detection procedures in FIGS. 4(a), 4(b), 5(a) and 5(b). The detection results are shown as follows.

When the first detection model corresponding to the time interval of 4 seconds was adopted, 288 electrocardiogram signal segments were falsely determined as auricular fibrillation among 7223 normal electrocardiogram signal segments, and 1314 electrocardiogram signal segments were falsely determined as normal electrocardiogram signal segments among 1982 electrocardiogram signal segments of auricular fibrillation. In other words, an overall detection precision rate is 82.6%, and the positioning accuracy is 4 seconds.

When the second detection model corresponding to the time interval of 8 seconds was adopted, 115 electrocardiogram signal segments were falsely determined as auricular fibrillation among 3615 normal electrocardiogram signal segments, and 426 electrocardiogram signal segments were falsely determined as normal electrocardiogram signal segments among 987 electrocardiogram signal segments of auricular fibrillation. In other words, an overall detection precision rate is 88.2%, and the positioning accuracy is 8 seconds.

When the detection procedure in FIGS. 4(a) and 4(b) was adopted, the positioning accuracy for auricular fibrillation was 4 seconds, 216 electrocardiogram signal segments were falsely determined as auricular fibrillation among 7223 normal electrocardiogram signal segments, and 1023 electrocardiogram signal segments were falsely determined as normal electrocardiogram signal segments among 1982 electrocardiogram signal segments of auricular fibrillation. In other words, an overall detection precision rate is 86.5%.

When the detection procedure in FIGS. 5(a) and 5(b) was adopted, the positioning accuracy for auricular fibrillation was 4 seconds, 141 electrocardiogram signal segments were falsely determined as auricular fibrillation among 7223 normal electrocardiogram signal segments, and 1260 electrocardiogram signal segments were falsely determined as normal electrocardiogram signal segments among 1982 electrocardiogram signal segments of auricular fibrillation. In other words, an overall detection precision rate is 84.8%.

As mentioned above, through the auricular fibrillation detection procedures in the embodiments of the present disclosure, it is able to ensure the positioning accuracy of 4 seconds, and provide the detection precision rate obviously advantageous over that acquired by the first detection model corresponding to the time interval of 4 seconds.

The above detection procedures may each be implemented as a series of steps. However, the detection procedure may not necessarily be implemented through the steps in the embodiments of the present disclosure as well as a specific combination thereof. In the case of not departing from the principle of the present disclosure, the steps in the embodiments of the present disclosure may be combined, and the order of the steps may be adjusted. The detection procedures acquired after changing the combination of the steps and adjusting the order of the steps shall also fall within the scope of the present disclosure.

The present disclosure further provides in some embodiments an arrhythmia detection system 600 which, as shown in FIG. 6, includes: an electrocardiogram signal acquisition circuit 601 configured to acquire an electrocardiogram signal; a setting circuit 602 configured to set a group of time intervals with gradually increasing lengths, a later one of two adjacent time intervals having a length as an integral multiple of a former one of the two adjacent time intervals; a division circuit 603 configured to equally divide the electrocardiogram signal acquired by the electrocardiogram signal acquisition circuit 601 through each of the time intervals, so as to acquire sequences of electrocardiogram signal segments, each time interval in the group of time intervals corresponding to one sequence of electrocardiogram signal segments; a first memory 604 configured to store therein the sequences of electrocardiogram signal segments acquired by the division circuit 603; a determination circuit 605 configured to read each sequence of electrocardiogram signal segments from the first memory 604, and determine an arrhythmia probability for each electrocardiogram signal segment in the sequence of electrocardiogram signal segments; a second memory 606 configured to store therein the arrhythmia probability for each electrocardiogram signal segment in the each sequence of electrocardiogram signal segments determined by the determination circuit 605; and a detection circuit 607 configured to read each sequence of electrocardiogram signal segments from the first memory 604, read the arrhythmia probability for each electrocardiogram signal segment in each sequence of electrocardiogram signal segments from the second memory 606, and for two sequences of electrocardiogram signal segments corresponding to two adjacent time intervals, determine an arrhythmia detection result for each electrocardiogram signal segment in the sequence of electrocardiogram signal segments corresponding to a shorter one of the time intervals in accordance with the arrhythmia probability for each electrocardiogram signal segment in the sequence of electrocardiogram signal segments corresponding to the shorter one of the time intervals, and the arrhythmia probability or an arrhythmia detection result for each electrocardiogram signal segment in the sequence of electrocardiogram signal segments corresponding to a longer one of the time intervals and including the electrocardiogram signal segments in the sequence of electrocardiogram signal segments corresponding to the shorter one of the time intervals.

It should be appreciated that, units and steps described in the embodiments of the present disclosure may be implemented in the form of electronic hardware, or a combination of a computer program and the electronic hardware. Whether or not these functions are executed by hardware or software depends on specific applications or design constraints of the technical solution. Different methods may be adopted with respect to the specific applications so as to achieve the described functions, without departing from the scope of the present disclosure.

It should be further appreciated that, for convenience and clarification, operation procedures of the system, device and units described hereinabove may refer to the corresponding procedures in the method embodiments, and thus will not be particularly defined herein.

It should be further appreciated that, the device and method may be implemented in any other ways. For example, the embodiments for the apparatus are merely for illustrative purposes, and the modules or units are provided merely on the basis of their logic functions. During the actual application, some modules or units may be combined together or integrated into another system. Alternatively, some functions of the module or units may be omitted or not executed. In addition, the coupling connection, direct coupling connection or communication connection between the modules or units may be implemented via interfaces, and the indirect coupling connection or communication connection between the modules or units may be implemented in an electrical or mechanical form or in any other form.

The units may be, or may not be, physically separated from each other. The units for displaying may be, or may not be, physical units, i.e., they may be arranged at an identical position, or distributed on a plurality of network elements. Parts or all of the units may be selected in accordance with the practical need, so as to achieve the purpose of the present disclosure.

In addition, the functional units in the embodiments of the present disclosure may be integrated into a processing unit, or the functional units may exist independently, or two or more functional units may be combined together.

In the case that the functional units are implemented in a software form and sold or used as a separate product, they may be stored in a computer-readable medium. Based on this, the technical solutions of the present disclosure, partial or full, or parts of the technical solutions of the present disclosure contributing to the prior art, may appear in the form of software products, which may be stored in a storage medium and include several instructions so as to enable computer equipment (a personal computer, a server or network equipment) to execute all or parts of the steps of the method according to the embodiments of the present disclosure. The storage medium includes any medium capable of storing therein program codes, e.g., a universal serial bus (USB) flash disk, a mobile hard disk (HD), a read-only memory (ROM), a random access memory (RAM), a magnetic disk or an optical disk.

It should be further appreciated that, all or parts of the steps in the method may be implemented by related hardware using a program stored in a computer-readable storage medium. The program may be executed so as to perform the above-mentioned method. The storage medium includes a magnetic disk, an optical disk, a read-only memory (ROM), a random access memory (RAM), or the like.

The above embodiments are for illustrative purposes only, but the present disclosure is not limited thereto. Obviously, a person skilled in the art may make further modifications and improvements without departing from the spirit of the present disclosure, and these modifications and improvements shall also fall within the scope of the present disclosure.

What is claimed is:

1. An arrhythmia detection method, comprising acquiring an electrocardiogram signal;
setting a group of time intervals with gradually increasing lengths, wherein for each two adjacent time intervals in the group of time intervals, a length of a latter time interval is an integral multiple of a length of a former time interval;
equally dividing the acquired electrocardiogram signal according to each of the group of time intervals to acquire respective sequences of electrocardiogram signal segments, wherein each of the group of time intervals corresponds to a respective sequence of electrocardiogram signal segments;
determining an arrhythmia probability for each electrocardiogram signal segment in each of the sequences of electrocardiogram signal segments; and
for respective two sequences of electrocardiogram signal segments corresponding to each two adjacent time intervals, determining an arrhythmia detection result for each electrocardiogram signal segment in the sequence of electrocardiogram signal segments corresponding to a shorter one of the time intervals in accordance with the arrhythmia probability for the electrocardiogram signal segment in the sequence of electrocardiogram signal segments corresponding to the shorter one of the time intervals, and the arrhythmia probability or an arrhythmia detection result for a respective electrocardiogram signal segment in the sequence of electrocardiogram signal segments corresponding to a longer one of the time intervals, wherein the respective electrocardiogram signal segment in the sequence of electrocardiogram signal segments corresponding to the longer one of the time intervals comprises the electrocardiogram signal segment in the sequence of electrocardiogram signal segments corresponding to the shorter one of the time intervals.

2. The arrhythmia detection method according to claim 1, wherein for each two adjacent time intervals, a start position of each electrocardiogram signal segment in the sequence of electrocardiogram signal segments corresponding to the longer one of the time intervals is aligned with a start position of a respective one electrocardiogram signal segment in the sequence of electrocardiogram signal segments corresponding to the shorter one of the time intervals.

3. The arrhythmia detection method according to claim 1, wherein the arrhythmia detection result comprises a label indicating an arrhythmia condition.

4. The arrhythmia detection method according to claim 1, wherein for respective two sequences of electrocardiogram signal segments corresponding to each two adjacent time intervals, the determining the arrhythmia detection result for each electrocardiogram signal segment in the sequence of electrocardiogram signal segments corresponding to the shorter one of the time intervals comprises:
when the longer one of the time intervals is a maximum time interval in the groups of time intervals, determining the arrhythmia detection result for each electrocardiogram signal segment in the sequence of electrocardiogram signal segments corresponding to the shorter one of the time intervals in accordance with the arrhythmia probability for the electrocardiogram signal segment in the sequence of electrocardiogram signal segments corresponding to the shorter one of the time intervals, and the arrhythmia probability for the respective electrocardiogram signal segment in the sequence of electrocardiogram signal segments corresponding to the longer one of the time intervals, wherein the respective electrocardiogram signal segment in the sequence of electrocardiogram signal segments corresponding to the longer one of the time intervals comprises the electrocardiogram signal segment in the sequence of electrocardiogram signal segments corresponding to the shorter one of the time intervals, and
when the longer one of the time intervals is not the maximum time interval in the groups of time intervals, determining the arrhythmia detection result for each electrocardiogram signal segment in the sequence of electrocardiogram signal segments corresponding to the shorter one of the time intervals in accordance with the arrhythmia probability for the electrocardiogram signal segment in the sequence of electrocardiogram signal segments corresponding to the shorter one of the time intervals, and the arrhythmia detection result for the respective electrocardiogram signal segment in the sequence of electrocardiogram signal segments corresponding to the longer one of the time intervals, wherein the respective electrocardiogram signal segment in the sequence of electrocardiogram signal segments corresponding to the longer one of the time intervals comprises the electrocardiogram signal segment in the sequence of electrocardiogram signal segments corresponding to the shorter one of the time intervals.

5. The arrhythmia detection method according to claim 1, wherein the electrocardiogram signal segments in each sequence of electrocardiogram signal segments are consecutive.

6. An arrhythmia detection device, comprising a memory, a processor, and computer-executable instructions stored in the memory and configured to be executed by the processor, wherein the processor is configured to execute the computer-executable instructions to:
set a group of time intervals with gradually increasing lengths, wherein for each two adjacent time intervals in the group of time intervals, a length of a latter one time interval is an integral multiple of a length of a former one time interval;
equally divide an electrocardiogram signal through each of the time intervals to acquire respective sequences of electrocardiogram signal segments, wherein each of the time intervals in the group of time intervals corresponds to a respective one sequence of electrocardiogram signal segments;
determine an arrhythmia probability for each electrocardiogram signal segment in each of the sequences of electrocardiogram signal segments; and
for respective two sequences of electrocardiogram signal segments corresponding to each two adjacent time intervals, determine an arrhythmia detection result for each electrocardiogram signal segment in the sequence of electrocardiogram signal segments corresponding to a shorter one of the time intervals in accordance with the arrhythmia probability for the electrocardiogram signal segment in the sequence of electrocardiogram signal segments corresponding to the shorter one of the time intervals, and the arrhythmia probability or an arrhythmia detection result for a respective electrocardiogram signal segment in the sequence of electrocardiogram signal segments corresponding to a longer one of the time intervals, wherein the respective electrocardiogram signal segment in the sequence of electrocardiogram signal segments corresponding to the longer one of the time intervals comprises the electrocardiogram signal segment in the sequence of electrocardiogram signal segments corresponding to the shorter one of the time intervals.

7. The arrhythmia detection device according to claim 6, wherein for each two adjacent time intervals, a start position of each electrocardiogram signal segment in the sequence of electrocardiogram signal segments corresponding to the longer one of the time intervals is aligned with a start position of a respective one electrocardiogram signal segment in the sequence of electrocardiogram signal segments corresponding to the shorter one of the time intervals.

8. The arrhythmia detection device according to claim 6, wherein the arrhythmia detection result comprises a label indicating an arrhythmia condition.

9. The arrhythmia detection device according to claim 6, wherein the processor is further configured to execute the computer-executable instructions to, for respective two sequences of electrocardiogram signal segments corresponding to each two adjacent time intervals,
when the longer one of the time intervals is a maximum time interval in the groups of time intervals, determine the arrhythmia detection result for each electrocardiogram signal segment in the sequence of electrocardiogram signal segments corresponding to the shorter one of the time intervals in accordance with the arrhythmia probability for the electrocardiogram signal segment in the sequence of electrocardiogram signal segments corresponding to the shorter one of the time intervals, and the arrhythmia probability for a respective electrocardiogram signal segment in the sequence of electrocardiogram signal segments corresponding to the longer one of the time intervals, wherein the respective electrocardiogram signal segment in the sequence of electrocardiogram signal segments corresponding to the longer one of the time intervals comprises the electrocardiogram signal segment in the sequence of electrocardiogram signal segments corresponding to the shorter one of the time intervals, and
when the longer one of the time intervals is not the maximum time interval in the groups of time intervals, determine the arrhythmia detection result for each electrocardiogram signal segment in the sequence of electrocardiogram signal segments corresponding to the shorter one of the time intervals in accordance with the arrhythmia probability for the electrocardiogram signal segment in the sequence of electrocardiogram signal segments corresponding to the shorter one of the time intervals, and the arrhythmia detection result for the respective electrocardiogram signal segment in the sequence of electrocardiogram signal segments corresponding to the longer one of the time intervals, wherein the respective electrocardiogram signal segment in the sequence of electrocardiogram signal segments corresponding to the longer one of the time intervals comprises the electrocardiogram signal segment in the sequence of electrocardiogram signal segments corresponding to the shorter one of the time intervals.

10. The arrhythmia detection device according to claim 6, wherein the electrocardiogram signal segments in each sequence of electrocardiogram signal segments are consecutive.

11. The arrhythmia detection device according to claim 6, wherein the electrocardiogram signal segments in at least one of the sequences of electrocardiogram signal segments overlap each other.

12. The arrhythmia detection device according to claim 6, wherein the processor is further configured to execute the computer-executable instructions to, for each electrocardiogram signal segment in the sequence of electrocardiogram signal segments corresponding to the longer one of the time intervals in the respective two sequences of electrocardiogram signal segments corresponding to each two adjacent time intervals:
when the arrhythmia probability for the electrocardiogram signal segment is greater than or equal to a threshold, or when the arrhythmia detection result for the electrocardiogram signal segment is positive, and the arrhythmia probability for at least one of the electrocardiogram signal segments in the sequence of electrocardiogram signal segments corresponding to the shorter one of the time intervals comprised in the electrocardiogram signal segment in the sequence of electrocardiogram signal segments corresponding to the longer one of the time intervals is greater than the threshold, determine the arrhythmia detection result for each electrocardiogram signal segment in the sequence of electrocardiogram signal segments corresponding to the shorter one of the time intervals comprised in the electrocardiogram signal segment in the sequence of electrocardiogram signal segments corresponding to the longer one of the time intervals to be positive.

13. The arrhythmia detection device according to claim 10, wherein the processor is further configured to execute the computer-executable instructions to, for each electrocardiogram signal segment in the sequence of electrocardiogram signal segments corresponding to the longer one of the time intervals in the respective two sequences of electrocardiogram signal segments corresponding to each two adjacent time intervals:
when the arrhythmia probability for the electrocardiogram signal segment is greater than or equal to a threshold, or when the arrhythmia detection result for the electrocardiogram signal segment is positive, and the arrhythmia probability for each electrocardiogram signal segment in the sequence of electrocardiogram signal segments corresponding to the shorter one of the time intervals comprised in the electrocardiogram signal segment in the sequence of electrocardiogram signal segments corresponding to the longer one of the time intervals is smaller than the threshold, determine the arrhythmia detection result for at least one electrocardiogram signal segment, but not all the electrocardiogram signal segments, in the sequence of electrocardiogram signal segments corresponding to the shorter one of the time intervals comprised in the electrocardiogram signal segment in the sequence of electrocardiogram signal segments corresponding to the longer one of the time intervals to be positive.

14. The arrhythmia detection device according to claim 13, wherein the processor is further configured to execute the computer-executable instructions to, for each electrocardiogram signal segment in the sequence of electrocardiogram signal segments corresponding to the longer one of the time intervals in the respective two sequences of electrocardiogram signal segments corresponding to each two adjacent time intervals:
when the arrhythmia probability for the electrocardiogram signal segment is smaller than a threshold, or when the arrhythmia detection result for the electrocardiogram signal segment is negative, and the arrhythmia probability for at least one electrocardiogram signal segment in the sequence of electrocardiogram signal segments corresponding to the shorter one of the time intervals comprised in the electrocardiogram signal segment in the sequence of electrocardiogram signal segments corresponding to the longer one of the time intervals is smaller than the threshold, determine the arrhythmia detection result for each electrocardiogram signal segment in the sequence of electrocardiogram signal segments corresponding to the shorter one of the time intervals comprised in the electrocardiogram signal segment in the sequence of electrocardiogram signal segments corresponding to the longer one of the time intervals to be negative.

15. The arrhythmia detection device according to claim 13, wherein the processor is further configured to execute the computer-executable instructions to, for each electrocardiogram signal segment in the sequence of electrocardiogram signal segments corresponding to the longer one of the time intervals in the respective two sequences of electrocardiogram signal segments corresponding to each two adjacent time intervals:
when the arrhythmia probability for the electrocardiogram signal segment is smaller than a threshold, or when the arrhythmia detection result for the electrocardiogram signal segment is negative, and the arrhythmia probability for each electrocardiogram signal segment in the sequence of electrocardiogram signal segments corresponding to the shorter one of the time intervals comprised in the electrocardiogram signal segment in the sequence of electrocardiogram signal segments corresponding to the longer one of the time intervals is greater than or equal to the threshold, determine the arrhythmia detection result for at least one electrocardiogram signal segment, but not all the electrocardiogram signal segments, in the sequence of electrocardiogram signal segments corresponding to the shorter one of the time intervals comprised in the electrocardiogram signal segment in the sequence of electrocardiogram signal segments corresponding to the longer one of the time intervals to be negative.

16. The arrhythmia detection device according to claim 11, wherein the processor is further configured to execute the computer-executable instructions to, for each electrocardiogram signal segment in the sequence of electrocardiogram signal segments corresponding to the shorter one of the time intervals in the respective two sequences of electrocardiogram signal segments corresponding to each two adjacent time intervals:
when each of the arrhythmia probabilities for at least two electrocardiogram signal segments in the sequence of electrocardiogram signal segments corresponding to the longer one of the time intervals comprising the electrocardiogram signal segment in the sequence of electrocardiogram signal segments corresponding to the shorter one of the time intervals is greater than or equal to a threshold, or when the arrhythmia detection result for the electrocardiogram signal segment in the sequence of electrocardiogram signal segments corresponding to the shorter one of the time intervals is positive, determine the arrhythmia detection result for the electrocardiogram signal segment in the sequence of electrocardiogram signal segments corresponding to the shorter one of the time intervals to be positive.

17. The arrhythmia detection device according to claim 11, wherein the processor is further configured to execute the computer-executable instructions to, for each electrocardiogram signal segment in the sequence of electrocardiogram signal segments corresponding to the shorter one of the time intervals in the respective two sequences of electrocardiogram signal segments corresponding to each two adjacent time intervals:
when each of the arrhythmia probabilities for at least two electrocardiogram signal segments in the sequence of electrocardiogram signal segments corresponding to the longer one of the time intervals comprising the electrocardiogram signal segment in the sequence of electrocardiogram signal segments corresponding to the shorter one of the time intervals is smaller than a threshold, or when the arrhythmia detection result for each of the electrocardiogram signal segments in the sequence of electrocardiogram signal segments corresponding to the shorter one of the time intervals is negative, determine the arrhythmia detection result for the electrocardiogram signal segment in the sequence of electrocardiogram signal segments corresponding to the shorter one of the time intervals to be negative.

18. The arrhythmia detection device according to claim 17, wherein the processor is further configured to execute the computer-executable instructions to, for each electrocardiogram signal segment in the sequence of electrocardiogram signal segments corresponding to the shorter one of the time intervals in the respective two sequences of electrocardiogram signal segments corresponding to each two adjacent time intervals:
when the arrhythmia probabilities or arrhythmia detection results for at least two electrocardiogram signal segments in the sequence of electrocardiogram signal segments corresponding to the longer one of the time intervals comprising the electrocardiogram signal segment in the sequence of electrocardiogram signal segments corresponding to the shorter one of the time intervals are inconsistent with each other, determine the arrhythmia detection result for the electrocardiogram signal segment in the sequence of electrocardiogram signal segments corresponding to the shorter one of the time intervals in accordance with the arrhythmia probability for the electrocardiogram signal segment in the sequence of electrocardiogram signal segments corresponding to the shorter one of the time intervals.

19. The arrhythmia detection device according to claim 6, wherein the group of time intervals comprises a first time interval and a second time interval, a length of the second time interval is twice of a length of the first time interval, the first time interval corresponds to a first sequence of electrocardiogram signal segments, and the second time interval corresponds to a second sequence of electrocardiogram signal segments,
wherein the processor is further configured to execute the computer-executable instructions to, for merely the first sequence of electrocardiogram signal segments and the second sequence of electrocardiogram signal segments, determine the arrhythmia detection result for each electrocardiogram signal segment in the first sequence of electrocardiogram signal segments in accordance with the arrhythmia probability for the electrocardiogram signal segment in the first sequence of electrocardiogram signal segments, and the arrhythmia probability for the respective electrocardiogram signal segment in the second sequence of electrocardiogram signal segments, wherein the respective electrocardiogram signal segment in the second sequence of electrocardiogram signal segments comprises the electrocardiogram signal segment in the first sequence of electrocardiogram signal segments.

20. An arrhythmia detection system, comprising:
an electrocardiogram signal acquisition circuit configured to acquire an electrocardiogram signal;
a setting circuit configured to set a group of time intervals with gradually increasing lengths, wherein for each two adjacent time intervals in the group of time intervals, a length of a latter one time interval is an integral multiple of a length of a former one time interval;
a division circuit configured to equally divide the electrocardiogram signal acquired by the electrocardiogram signal acquisition circuit through each of the time intervals to acquire respective sequences of electrocardiogram signal segments, wherein each of the time intervals in the group of time intervals corresponds to a respective one sequence of electrocardiogram signal segments;
a first memory configured to store therein the sequences of electrocardiogram signal segments acquired by the division circuit;
a determination circuit configured to read each of the sequences of electrocardiogram signal segments from the first memory, and determine an arrhythmia probability for each electrocardiogram signal segment in each of the sequences of electrocardiogram signal segments;
a second memory configured to store therein the arrhythmia probability for each electrocardiogram signal segment in the each sequence of electrocardiogram signal segments determined by the determination circuit; and
a detection circuit configured to read each of the sequences of electrocardiogram signal segments from the first memory, read the arrhythmia probability for each electrocardiogram signal segment in each of the sequences of electrocardiogram signal segments from the second memory, and for respective two sequences of electrocardiogram signal segments corresponding to each two adjacent time intervals, determine an arrhythmia detection result for each electrocardiogram signal segment in the sequence of electrocardiogram signal segments corresponding to a shorter one of the time intervals in accordance with the arrhythmia probability for the electrocardiogram signal segment in the sequence of electrocardiogram signal segments corresponding to the shorter one of the time intervals, and the arrhythmia probability or an arrhythmia detection result for a respective electrocardiogram signal segment in the sequence of electrocardiogram signal segments corresponding to a longer one of the time intervals, wherein the respective electrocardiogram signal segment in the sequence of electrocardiogram signal segments corresponding to the longer one of the time intervals comprises the electrocardiogram signal segment in the sequence of electrocardiogram signal segments corresponding to the shorter one of the time intervals.
